(19) United States
(12) Patent Application Publication
(10) Pub. No.: US 2017/0027452 A1
(43) Pub. Date: Feb. 2, 2017

(54) WAVEFORM ANALYSIS METHOD, WAVEFORM ANALYSIS APPARATUS, AND COMPUTER READABLE STORAGE MEDIUM STORING WAVEFORM ANALYSIS PROGRAM

(71) Applicant: Nihon Kohden Corporation, Tokyo (JP)

(72) Inventors: Yoshinobu Ono, Tokyo (JP); Tsuneo Takayanagi, Tokyo (JP); Takashi Kaiami, Tokyo (JP)

(21) Appl. No.: 15/220,626

(22) Filed: Jul. 27, 2016

(30) Foreign Application Priority Data
Jul. 28, 2015 (JP) 2015-148722

(51) Int. Cl. A61B 5/0205; A61B 5/04; A61B 5/0452

(52) U.S. Cl. CPC A61B 5/0205; A61B 5/0452; A61B 5/04012; A61B 5/0816

(57) ABSTRACT

A waveform analysis method for analyzing vital sign waveform data by using respiratory waveform data which are synchronized with the vital sign waveform data includes acquiring the vital sign waveform data and the respiratory waveform data, determining an expiratory phase and an inspiratory phase from the respiratory waveform data, extracting given waveform data corresponding to the expiratory phase, and given waveform data corresponding to the inspiratory phase from the vital sign waveform data, performing a first waveform analysis M which a given waveform analysis is applied to the extracted given waveform data corresponding to the expiratory phase, and performing a second waveform analyzing in which the given waveform analysis is applied to the extracted given waveform data corresponding to the inspiratory phase.

### CROSS REFERENCE TO RELATED APPLICATION

This application is based on Japanese Patent Applications No. 2015-148722 filed on Jul. 28, 2015, the contents of which are incorporated herein by reference.

### BACKGROUND

The presently disclosed subject matter relates to a method for analyzing a waveform (hereinafter, referred to as a waveform analysis method), and also to an apparatus for analyzing a waveform (hereinafter, referred to as a waveform analysis apparatus), a program for analyzing a waveform (hereinafter, referred to as a waveform analysis program), and a computer readable storage medium on which the waveform analysis program is stored.

Usually, respiratory variation which is caused by respiration of the patient is superimposed on a vital sign waveform such as an electrocardiogram waveform, and the amplitudes of the QRS wave, T-wave, and the like of the electrocardiogram waveform are affected by the respiratory variation. When a vital sign waveform such as an electrocardiogram waveform on which respiratory variation is superimposed is to be analyzed, therefore, a filtering process of removing waveform components of frequencies corresponding to respiratory variation from the vital sign waveform is performed before the waveform analysis (for example, see Japanese Patent No. 3,877,761). In the filtering process disclosed in Japanese Patent No. 3,877,761, respiratory variation may be removed from a vital sign waveform, but the original shape of the vital sign waveform is changed. The shape change of the original waveform affects the width and amplitude of the vital sign waveform during the waveform analysis. Therefore, there is a possibility that the change may affect a result of diagnosis on the patient in which the waveform analysis is used. Moreover, there is a further possibility that respiratory variation may not be completely removed from the vital sign waveform by the filtering process.

It is an object of the presently disclosed subject matter to provide a waveform analysis method and apparatus which, without changing the shape of the original waveform of vital sign waveform data, may analyze the vital sign waveform data after consideration of an influence of respiratory variation, It is another object of the presently disclosed subject matter to provide a waveform analysis program which is used for realizing the waveform analysis method, and a computer readable storage medium on which the waveform analysis program is stored.

### SUMMARY

According to an aspect of the presently disclosed subject matter, a waveform analysis method for analyzing vital sign waveform data by using respiratory waveform data which are synchronized with the vital sign waveform data includes acquiring the vital sign waveform data and the respiratory waveform data, determining an expiratory phase and an inspiratory phase from the respiratory waveform data, extracting given waveform data corresponding to the expiratory phase, and given waveform data corresponding to the inspiratory phase from the vital sign waveform data, performing a first waveform analysis in which a given waveform analysis is applied to the extracted given waveform data corresponding to the expiratory phase, and performing a second waveform analyzing in which the given waveform analysis is applied to the extracted given waveform data corresponding to the inspiratory phase.

Therefore, it is possible to provide a waveform analysis method which, without changing the shape of the original waveform of vital sign waveform data, may analyze the vital sign waveform data after consideration of an influence of respiratory variation.

According to the waveform analysis method, moreover, the original waveform of the vital sign waveform data is not changed by a filtering process or the like. Therefore, vital sign information which is more accurate than conventional one, and novel vital sign information which does not conventionally exist may be acquired from the vital sign waveform data.

### BRIEF DESCRIPTION OF DRAWINGS

FIG. 1 is a hardware configuration diagram illustrating a waveform analysis apparatus of an embodiment of the presently disclosed subject matter.

FIG. 2 is a functional block diagram of a controller.

FIG. 3A illustrates an electrocardiogram waveform on which respiratory variation is superimposed, and FIG. 3B illustrates a respiratory waveform.

FIG. 4 is a flowchart illustrating a TWA analysis in which the waveform analysis apparatus of the embodiment of the presently disclosed subject matter is used.

FIG. 5 is a flowchart illustrating a J-wave analysis in which the waveform analysis apparatus of the embodiment of the presently disclosed subject matter is used.

FIG. 6 is a flowchart illustrating an alternans analysis in which the waveform analysis apparatus of the embodiment of the presently disclosed subject matter is used.

### DETAILED DESCRIPTION OF EMBODIMENTS

Hereinafter, an embodiment of the presently disclosed subject matter will be described with reference to the drawings. In the description of the embodiment, description of components which are denoted by the same reference numerals as those designating components that have been already described will be omitted for the sake of convenience of description.

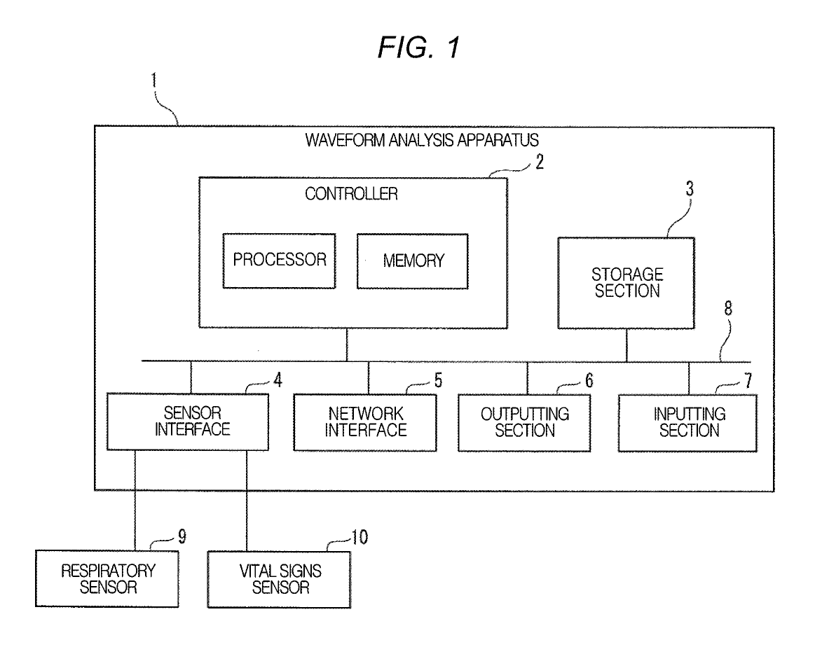

FIG. 1 is a hardware configuration diagram of a waveform analysis apparatus 1 of the embodiment of the presently disclosed subject matter. As illustrated in FIG. 1, the waveform analysis apparatus 1 may include a controller 2, a storage section 3, a sensor interface 4, a network interface 5, an outputting section 6, and an inputting section 7. These components are communicably connected to one another through a bus 8.

The waveform analysis apparatus 1 is an apparatus dedicated to waveform analysis, but alternatively may be a portable or wearable device such as a personal computer, a smartphone, a tablet, or an Apple Watch.

The controller 2 may include a memory and a processor. For example, the memory is configured by a ROM (Read Only Memory) in which various programs and the like are stored, a RAM (Random Access Memory) having a plurality of work areas in which various programs that are to be executed by the processor, and the like are to be stored. For example, the processor is a CPU (Central Processing Unit), and configured so as to develop designated programs in the various programs incorporated in the ROM, in the RAM, and execute various processes in cooperation with the RAM.

Particularly, the processor may develop a waveform analysis program which will be described later, in the RAM, and cooperate with the RAM to execute the waveform analysis program, thereby enabling the controller 2 to control various operations of the waveform analysis apparatus 1. The controller 2 and the waveform analysis program will be described in detail later.

The storage section 3 is a storage device such as an HDD (Hard Disk Drive), an SSD (Solid State Drive), or a flash memory, and configured so as to store programs and various data. The waveform analysis may be incorporated in the storage section 3. Moreover, respiratory waveform data acquired by a respiratory sensor 9, and vital sign waveform data (electrocardiogram waveform data, pulse wave waveform data, or the like) acquired by a vital signs sensor 10 may be stored in the storage section 3.

The sensor interface 4 communicably connects the waveform analysis apparatus 1 to the respiratory sensor 9 and the vital signs sensor 10. For example, the respiratory waveform data acquired by the respiratory sensor 9, and the vital sign waveform data acquired by the vital signs sensor 10 may be transmitted to the controller 2 through the sensor interface 4. The sensor interface 4 may be provided with an A/D function.

The network interface 5 connects the waveform analysis apparatus 1 to a communication network which is not illustrated. The communication network includes a LAN (Local Area Network), a WAN (Wide Area Network), the Internet, or the like. For example, an analysis result output from the controller 2 may be transmitted to another computer placed on the communication network through the network interface 5.

The outputting section 6 includes a display device such as a liquid crystal display or an organic EL display, and a print apparatus such as an inkjet printer or a laser printer. For example, the analysis result output from the controller 2 may be displayed on the display screen of the display device, or printed by the printer.

The inputting section 7 receives an input operation performed by the operator who operates the waveform analysis apparatus 1, and outputs an operation signal in accordance with the input operation. The inputting section 7 is a touch panel which is overlaid on the display device of the outputting section 6, operation buttons which are attached to a housing, a mouse, a keyboard, or the like.

The respiratory sensor 9 and the vital signs sensor 10 are to be attached to the same patient. The respiratory sensor 9 measures the motion of the lungs of the patient, and the flow rate and air pressure of the breath through the mouth and nose of the patient, to acquire the respiratory waveform data of the patient. The vital sips sensor 10 measures a physical quantity obtained from the patient, to acquire vital sign waveform data of the patient. The vital signs sensor 10 has a concept in which a respiratory sensor is not included, and the vital signs sensor 10 and the respiratory sensor 9 are different devices. In the case where the vital signs sensor 10 is an electrocardiogram sensor, particularly, the electrocardiogram sensor measures a weak electrical signal produced by the heart of the patient, to acquire electrocardiogram waveform data. In the case where the vital signs sensor 10 is a pulse wave sensor, the pulse wave sensor measures the absorption amount of a light beam which is emitted toward the blood vessel of the patient, to acquire pulse wave waveform data.

The controller 2 of the waveform analysis apparatus 1 of the embodiment analyzes vital sign waveform data by using respiratory waveform data which are synchronized with the vital sign waveform data. Therefore, the driving time of the respiratory sensor 9 overlaps with that of the vital signs sensor 10.

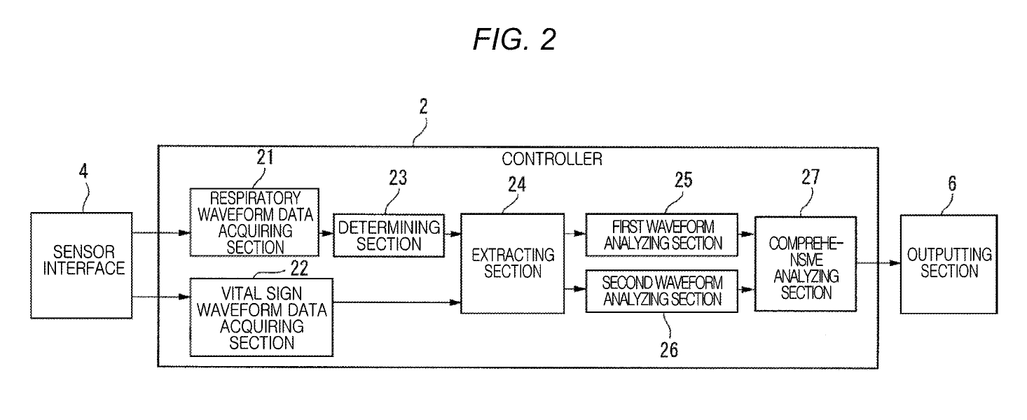

FIG. 2 is a diagram illustrating functional blocks of the controller 2 of the waveform analysis apparatus 1 illustrated in FIG. 1. As illustrated in FIG. 2, the controller 2 may include a respiratory waveform data acquiring section 21, a vital sign waveform data acquiring section 22, a determining section 23, an extracting section 24, a first waveform analyzing section 25, a second waveform analyzing section 26, and a comprehensive analyzing section 27.

The respiratory waveform data acquiring section 21 acquires the respiratory waveform data acquired by the respiratory sensor 9, through the sensor interface 4. The vital sign waveform data acquiring section 22 acquires the vital sign waveform data (for example, electrocardiogram waveform data or pulse wave waveform data) acquired by the vital signs sensor 10, through the sensor interface 4.

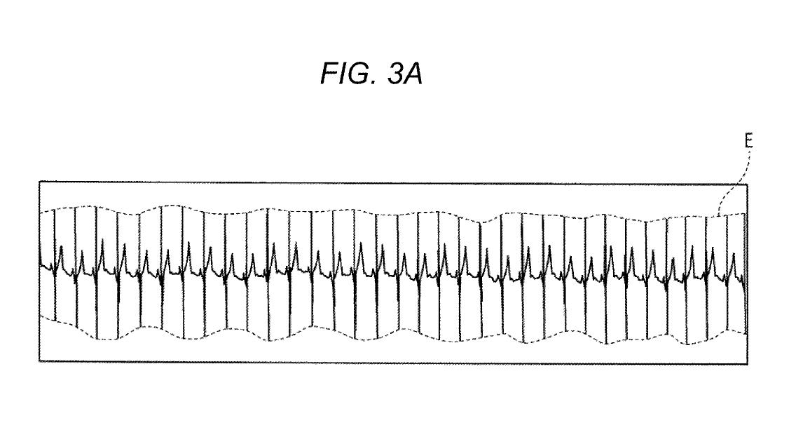

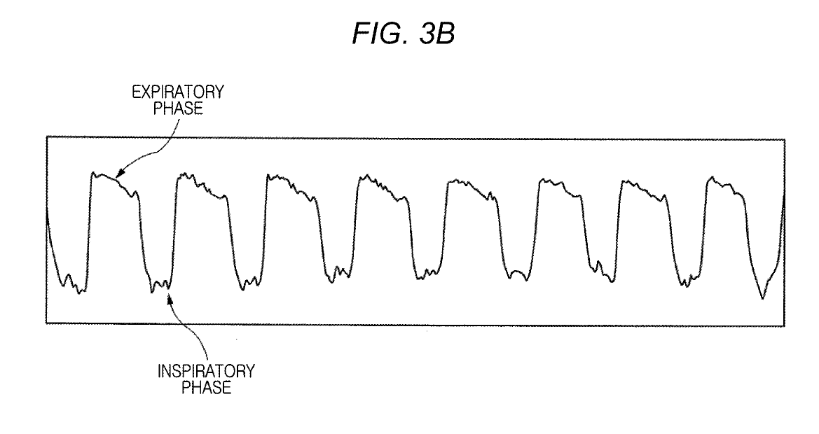

As illustrated in FIG. 3A, respiratory variation is superimposed on the acquired vital sign waveform data. FIG. 3A illustrates an electrocardiogram waveform that is an example of the vital sign waveform data on which respiratory variation is superimposed, and FIG. 3B illustrates a respiratory waveform. Both the abscissas of the electrocardiogram waveform and the respiratory waveform are time axes. In the respiratory waveform, as illustrated in FIG. 3B, the expiratory phase and the inspiratory phase are alternately repeated, a peak of the respiratory waveform corresponds to the expiratory phase, and a valley of the respiratory waveform corresponds to the inspiratory phase. As illustrated in FIG. 3A, it will be understood from the envelope E of the electrocardiogram waveform that the electrocardiogram waveform is fluctuated by the expiratory and inspiratory phases which are alternately repeated, In the embodiment, the vital sign waveform data are analyzed after consideration of an influence of respiratory variation which is caused by such respiration of the patient.

The determining section 23 determines the expiratory phase and the inspiratory phase (see FIG. 3B) from the respiratory waveform data which are acquired by the respiratory waveform data acquiring section 21. As described above, a peak of the respiratory waveform corresponds to the expiratory phase, and a valley of the respiratory waveform corresponds to the inspiratory phase (see FIG. 3B). Therefore, the determining section 23 may determine the expiratory phase and the inspiratory phase based on, for example, an amount of change of the respiratory waveform data per unit of time (a time differential value of the respiratory waveform data). The determining section 23 determines the time periods of the expiratory phase and the inspiratory phase.

The extracting section 24 extracts given waveform data corresponding to the expiratory phase, and given waveform data corresponding to the inspiratory phase (examples of the given waveform data are the P-wave, QRS wave, T-wave, J-wave, ant the like of the electrocardiogram waveform) from the vital sign waveform data acquired by the vital sign waveform data acquiring section 22. By using respiratory waveform data which are synchronized (coincident in time) with the vital sign waveform data Here, the extracting section 24 extracts a plurality of given waveform data which overlap with the time periods of the expiratory phase, and a plurality of given waveform data which overlap with the time periods of the inspiratory phase.

The first waveform analyzing section 25 performs a given waveform analysis (for example, an electrocardiogram analysis, or an alternans analysis) on the extracted given waveform data corresponding to the expiratory phase. The second waveform analyzing section 26 performs a similar given waveform analysis on the extracted given waveform data corresponding to the inspiratory phase.

The comprehensive analyzing section 27 comprehensively analyzes the vital sign waveform data based on an analysis result obtained by the first waveform analyzing section 25, and an analysis result obtained by the second waveform analyzing section 26. The outputting section 6 outputs an analysis result of the comprehensive analyzing section 27 by means of displaying/priming.

According to the embodiment, the respiratory waveform data which are synchronized with the vital sign waveform data are used, and therefore given waveform data corresponding to the expiratory phase, and those corresponding to the inspiratory phase are extracted from the vital sign waveform data. Moreover, the given waveform analysis is performed on the extracted waveform data corresponding to the expiratory phase, and the given waveform analysis is performed on the extracted waveform data corresponding to the inspiratory phase. Namely, waveform data of the expiratory phase are separated from those of the inspiratory phase, and therefore an error in the waveform analysis which is caused by mixture of waveform data of the expiratory and inspiratory phases may be eliminated.

Therefore, it is possible to provide the waveform analysis apparatus 1 which, without changing the shape of the original waveform of vital sign waveform data, may analyze the vital sign waveform data after consideration of an influence of respiratory variation.

In the waveform analysis apparatus 1, moreover, the original waveform of the vital sign waveform data is not changed by a filtering process or the like. Therefore, vital sign information which is more accurate than conventional one, and novel vital sign information which does not conventionally exist may be acquired from vital sign waveform data.

### ANALYSIS EXAMPLE 1

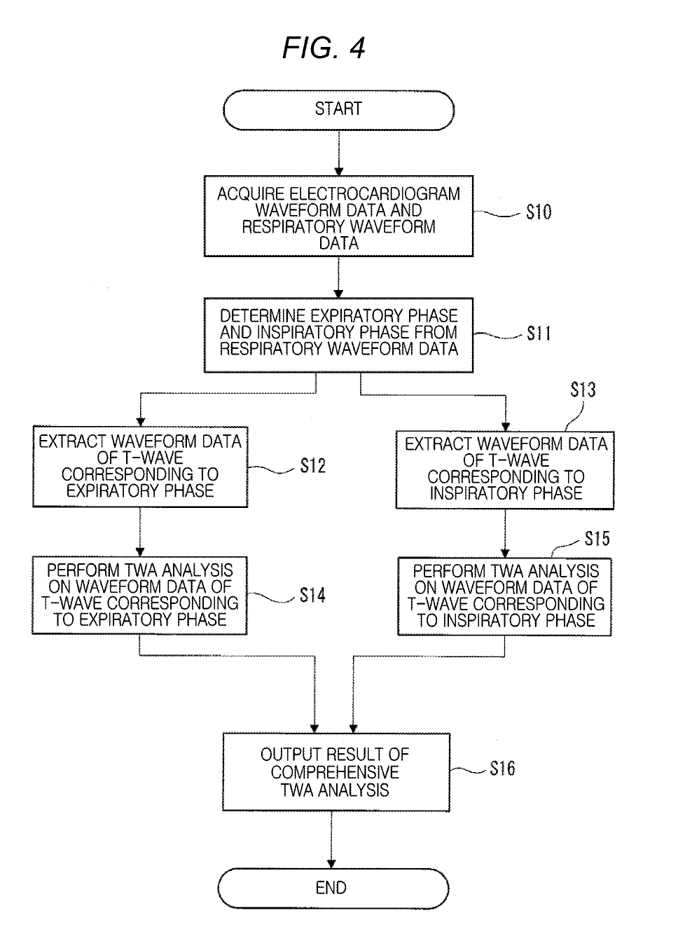

Next, a TWA analysis of electrocardiogram waveform data in which the waveform analysis apparatus 1 is used will be described with reference to FIG. 4. FIG. 4 is a flowchart illustrating the TWA analysis in which the waveform analysis apparatus 1 is used.

Here, a TWA (T-Wave Alternans) appears at onset of illness such as long QT syndrome, variant angina, acute myocardial ischemia, electrolyte imbalance, paroxysmal atrial tachycardia, bradycardia, or pericardial effusion. A TWA is a phenomenon in which the amplitude and polarity of the T-wave appearing in an electrocardiogram are alternately changed, and an index effective to predic sudden cardiac death. A TWA is not a phenomenon which may be always observed with the naked eye, and is therefore susceptible to be affected by respiratory variation. With respect to a TWA analysis, refer to, for example, JP-A-2015-112460.

In an electrocardiogram waveform on which respiratory variation is superimposed, there is a case where one of a pair of T-waves is in the expiratory phase, and the other of the pair of the T-waves is in the inspiratory phase. As the difference between the amplitudes of adjacent T-waves is measured in TWA analysis, it may be susceptible to respiratory variation. Moreover, the frequency of respiratory variation is close to the occurrence frequency of the T-wave. In the case where respiratory variation is removed from the electrocardiogram waveform by a filtering process or the like, there is a possibility that the shape of the T-wave may be largely changed.

In the waveform analysis apparatus I of the embodiment, by contrast, without changing the shape of the original waveform (including the shape of the T-wave) of the electrocardiogram waveform data, a TWA analysis may be performed after consideration of an influence of respiratory variation, as described below.

In step S10, firstly, the respiratory waveform data acquiring section 21 acquires the respiratory waveform data from the respiratory sensor 9 through the sensor interface 4, and the vital sign waveform data acquiring section 22 acquires the electrocardiogram waveform data from the vital signs sensor 10 through the sensor interface 4.

In step S11, next, the determining section 23 determines the expiratory phase and the inspiratory phase from the acquired respiratory waveform data. Thereafter, the extracting section 24 extracts waveform data of the T-wave corresponding to the expiratory phase (step S12), and extracts waveform data of the T-wave corresponding to the inspiratory phase (step S13).

Then, the first waveform analyzing section 25 performs a TWA analysis on the extracted waveform data of the T-wave corresponding to the expiratory phase (step S14). On the other hand, the second waveform analyzing section 26 performs a TWA analysis on the extracted waveform data of the T-wave corresponding to the inspiratory phase (step S15). In the TWA analysis, the difference between the amplitudes of adjacent T-waves is measured. In step S16, then, the comprehensive analyzing section 27 comprehensively analyzes the T-wave based on an analysis result of the T-wave obtained by the first waveform analyzing section 25, and an analysis result of the T-wave obtained by the second waveform analyzing section 26, and the outputting section 6 outputs an analysis result of the comprehensive analyzing section 27 by means of displaying/printing.

In the embodiment, as described above, the TWA analysis is performed on the waveform data of the T-wave corresponding to the expiratory phase, and the TWA analysis is performed on the waveform data of the T-wave corresponding to the inspiratory phase. Namely, the waveform data of the T-wave of the expiratory phase are separated from those of the T-wave of the inspiratory phase, and therefore an error in the TWA analysis which is caused by mixture of waveform data of the T-wave of the expiratory phase and those of the T-wave of the inspiratory phase may be eliminated. Therefore, a TWA analysis may be performed without being affected by respiratory variation.

### ANALYSIS EXAMPLE 2

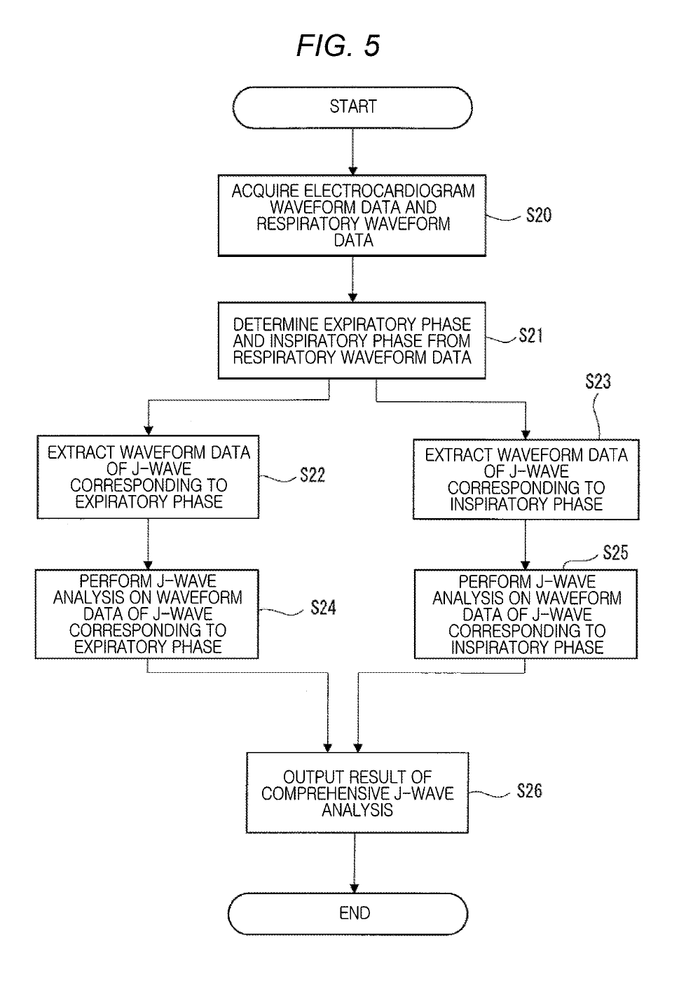

Next, a J-wave analysis of electrocardiogram waveform data in which the waveform analysis apparatus 1 is used will be described with reference to FIG. 5. FIG. 5 is a flowchart illustrating the J-wave analysis in which the waveform analysis apparatus 1 is used.

Here a J-wave is frequently observed in an electrocardiogram of a patient with idiopathic ventricular fibrillation. Therefore, a J-wave analysis has been studied in order to predict idiopathic ventricular fibrillation in a patient. A notch or slur shaped J-wave is known. It is determined whether the amplitude of such a J-wave satisfies a given threshold or not. On the other hand, the amplitude of an electrocardiogram waveform is easily varied by respiration. Therefore, also a J-wave analysis in which the amplitude of a J-wave is measured is susceptible to be affected by respiratory variation.

In the waveform analysis apparatus 1 of the embodiment, by contrast, without changing the shape of the original waveform (including the shape of the J-wave) of the electrocardiogram waveform data, the J-wave analysis may be performed after consideration of an influence of respiratory variation, as described below.

In step S20, firstly, the respiratory waveform data acquiring section 21 acquires the respiratory waveform data from the respiratory sensor 9 through the sensor interface 4, and the vital sign waveform data acquiring section 22 acquires the electrocardiogram waveform data from the vital signs sensor 10 through the sensor interface 4.

In step S21, next, the determining section 23 determines the expiratory phase and the inspiratory phase from the acquired respiratory waveform data. Thereafter, the extracting section 24 extracts waveform data of the J-wave corresponding to the expiratory phase (step S22), and extracts waveform data of the J-wave corresponding to the inspiratory phase (step S23).

Then, the first waveform analyzing section 25 performs a J-wave analysis on the extracted waveform data of the J-wave corresponding to the expiratory phase (step S24). On the other hand, the second waveform analyzing section 26 performs a J-wave analysis on the extracted waveform data of the J-wave corresponding to the inspiratory phase (step S25). In the J-wave analysis, it is determined whether the amplitude of the J-wave satisfies a given threshold or not. Moreover, different thresholds are set for the expiratory phase and the inspiratory phase, respectively. The threshold which is set for the expiratory phase is larger than that which is set for the inspiratory phase.

In step S26, then, the comprehensive analyzing section 27 comprehensively analyzes the J-wave based on an analysis result of the J-wave obtained by the first waveform analyzing section 25, and an analysis result of the J-wave obtained by the second waveform analyzing section 26, and the outputting section 6 outputs an analysis result of the comprehensive analyzing section 27 by means of displaying/printing.

In the embodiment, as described above, the J-wave analysis is performed on the waveform data of the J-wave corresponding to the expiratory phase, and the J-wave analysis is performed on the waveform data of the J-wave corresponding to the inspiratory phase. Namely, the waveform data of the J-wave of the expiratory phase are separated from those of the J-wave of the inspiratory phase, and therefore an error in the J-wave analysis which is caused by mixture of waveform data of the J-wave of the expiratory phase and those of the J-wave of the inspiratory phase may be eliminated. Therefore, a J-wave analysis may be performed without being affected by respiratory variation.

### ANALYSIS EXAMPLE 3

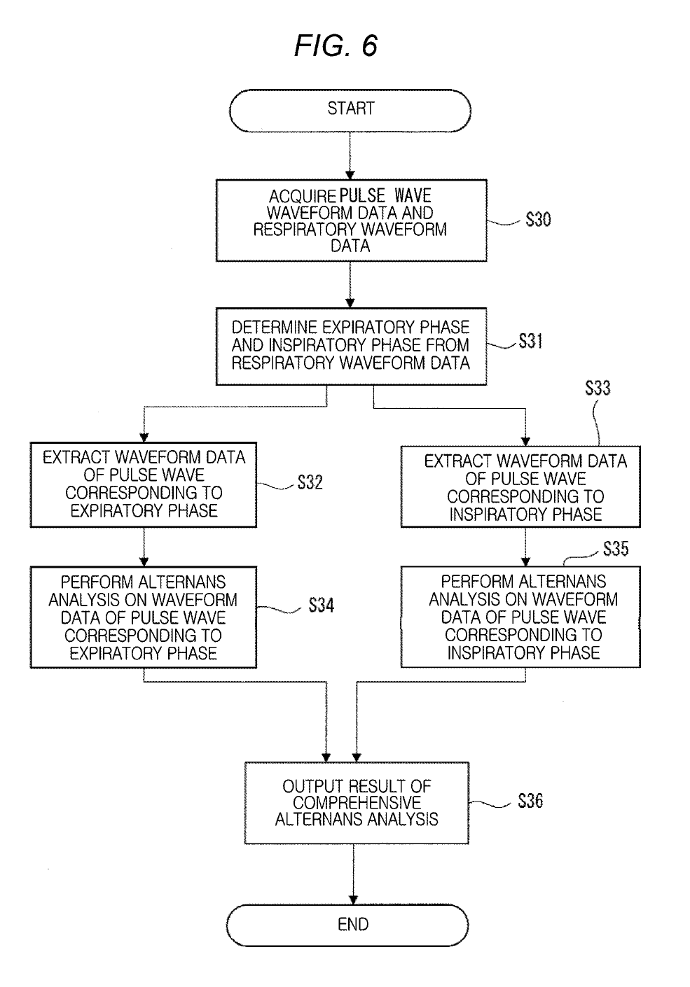

Next, an alternans analysis of electrocardiogram waveform data in which the waveform analysis apparatus 1 is used will be described with reference to FIG. 6. FIG. 6 is a flowchart illustrating the alternans analysis in which the waveform analysis apparatus 1 is used.

Here, an alternans is a phenomenon in which the amplitude of a pulse wave is alternated between low and high levels. It is reported that an alternans is a phenomenon which appears at the end stage of severe heart failure, and also that an alternans portends heart failure death or sudden death. Therefore, an alternans analysis has been studied.

In the waveform analysis apparatus 1 of the embodiment, by contrast, without changing the shape of the original waveform of the pulse wave waveform data, an alternans analysis may be performed after consideration of an influence of respiratory variation, as described below.

In step S30, firstly, the respiratory waveform data acquiring section 21 acquires the respiratory waveform data from the respiratory sensor 9 through the sensor interface 4, and the vital sign waveform data acquiring section 22 acquires the pulse wave waveform data from the vital signs sensor 10 through the sensor interface 4.

In step S31, next, the determining section 23 determines the expiratory phase and the inspiratory phase from the acquired respiratory waveform data. Thereafter, the extracting section 24 extracts waveform data of the pulse wave corresponding to the expiratory phase (step S32), and extracts waveform data of the pulse wave corresponding to the inspiratory phase (step S33).

Then, the first waveform analyzing section 25 performs an alternans analysis on the extracted waveform data of the pulse wave corresponding to the expiratory phase (step S34). On the other hand, the second waveform analyzing section 26 performs an alternans analysis on the extracted waveform data of the pulse wave corresponding to the inspiratory phase (step S35). In the alternans analysis, the difference between the amplitudes of adjacent pulse waves is measured.

In step S36, then, the comprehensive analyzing section 27 comprehensively analyzes the pulse wave based on an analysis result of the pulse wave obtained by the first waveform analyzing section 25, and an analysis result of the pulse wave obtained by the second waveform analyzing section 26, and the outputting section 6 outputs an analysis result of the comprehensive analyzing section 27 by means of displaying/printing.

In the embodiment, as described above, the alternans analysis is performed on the waveform data of the pulse wave corresponding to the expiratory phase, and the alternans analysis is performed on the waveform data of the pulse wave corresponding to the inspiratory phase. Namely, the waveform data of the pulse wave of the expiratory phase are separated from those of the pulse wave of the inspiratory phase, and therefore an error in the alternans analysis which is caused by mixture of waveform data of the pulse wave of the expiratory phase and those of the pulse wave of the inspiratory phase may be eliminated. Therefore, an alternans analysis may be performed without being affected by respiratory variation.

In order to realize the waveform analysis apparatus 1 of the embodiment in software, the waveform analysis program may be pre-installed in the storage section 3 or the ROM. Alternatively, the waveform analysis program may be stored on a computer readable storage medium such as a magnetic disk (an HDD or a floppy disk), an optical disk (a CD-ROM, a DVD-ROM, a Blu-ray disk, or the like), a magneto-optical disk (an MD or the like), a flash memory (an SD card, a USB memory, an SSD, or the like). In the alternative, when the storage medium is connected to the waveform analysis apparatus 1, the waveform analysis program which is stored in the storage medium is incorporated into the storage section 3. Then, the program incorporated in the storage section 3 is loaded into the RAM, the processor executes the loaded program, and as a result the controller 2 executes the various processes illustrated in FIG. 2. In other words, when the program is executed by the processor, the controller 2 functions as the respiratory waveform data acquiring section 21, the vital sign waveform data acquiring section 22, the determining section 23, the extracting section 24, the first waveform analyzing section 25, the second waveform analyzing section 26, and the comprehensive analyzing section 27.

Alternatively, the waveform analysis program may be downloaded from a computer on a communication network, through the network interface 5. Also in the alternative, similarly, the downloaded program is incorporated into the storage section 3.

Although the embodiment of the presently disclosed subject matter has been described, it is a matter of course that the technical scope of the presently disclosed subject matter should not be limitedly interpreted by the description of the embodiment. It should be understood by those skilled in the art that the embodiment is a mere example, and may be variously changed within the scope of the presently disclosed subject matter as defined in the claims. The technical scope of the presently disclosed subject matter should be determined based on the scope of the presently disclosed subject matter as defined in the claims, and the scope of equivalence thereof.

Although, in the embodiment, electrocardiogram waveform data and pulse wave waveform data have been described as the vital sign waveform data, the embodiment is not limited to this. However, the kind is not particularly limited as far as the vital sign waveform data has periodicity.

Although, in the embodiment, the analysis examples of T- and J-waves of electrocardiogram waveform data have been described, the embodiment is not limited to this. For example, the waveform analysis apparatus 1 of the embodiment may be applied also to an analysis of a waveform such as the QRS wave of an electrocardiogram waveform.

As described above, the waveform analysis apparatus 1 of the embodiment may be applied to arbitrary waveform analyses of vital sign waveform data of various kinds.

What is claimed is:

1. A waveform analysis method for analyzing vital sign waveform data by using respiratory waveform data which are synchronized with the vital sign waveform data, the method comprising:
acquiring the vital sign waveform data and the respiratory waveform data;
determining an expiratory phase and an inspiratory phase from the respiratory waveform data;
extracting given waveform data corresponding to the expiratory phase, and given waveform data corresponding to the inspiratory phase from the vital sign waveform data;
performing a first waveform analysis in which a given waveform analysis is applied to the extracted given waveform data corresponding to the expiratory phase; and
performing a second waveform analyzing in which the given waveform analysis is applied to the extracted given waveform data corresponding to the inspiratory phase.

2. The waveform analysis method according to claim 1, wherein the vital sign waveform data are electrocardiogram waveform data.

3. The waveform analysis method according to claim 2, wherein, in the extraction, waveform data of a T-wave corresponding to the expiratory phase, and waveform data of a T-wave corresponding to the inspiratory phase are extracted from the electrocardiogram waveform data,
in the first waveform analysis, a TWA analysis is performed on the extracted waveform data of the T-wave corresponding to the expiratory phase, and,
in the second waveform analysis. the TWA analysis is performed on the extracted waveform data of the T-wave corresponding to the inspiratory phase.

4. The waveform analysis method according to claim 2, wherein, in the extraction, waveform data of a J-wave corresponding to the expiratory phase, and waveform data of a J-wave corresponding to the inspiratory phase are extracted from the electrocardiogram waveform data,
in the first waveform analysis, a J-wave analysis is performed on the extracted waveform data of the J-wave corresponding to the expiratory phase, and,
in the second waveform analysis, the J-wave analysis is performed on the extracted waveform data of the J-wave corresponding to the inspiratory phase.

5. The waveform analysis method according to claim 1, wherein the vital sign waveform data are pulse wave waveform data,
in the extraction, waveform data of a pulse wave corresponding to the expiratory phase, and waveform data of a pulse wave corresponding to the inspiratory phase are extracted from the pulse wave waveform data,
in the first waveform analysis, an alternans analysis is performed on the extracted waveform data of the pulse wave corresponding to the expiratory phase, and,
in the second waveform analysis, the alternans analysis is performed on the extracted waveform data of the pulse wave corresponding to the inspiratory phase.

6. A waveform analysis apparatus for analyzing vital sign waveform data by using respiratory waveform data which are synchronized with the vital sign waveform data, the apparatus comprising:
a vital sign waveform data acquiring section that acquires the vital sign waveform data;
a respiratory waveform data acquiring section that acquires the respiratory waveform data;
a determining section that determines an expiratory phase and an inspiratory phase from the respiratory waveform data;
an extracting section that extracts given waveform data corresponding to the expiratory phase, and given waveform data corresponding to the inspiratory phase from the vital sign waveform data;
a first waveform analyzing section that performs a given waveform analysis on the extracted given waveform data corresponding to the expiratory phase; and
a second waveform analyzing section that performs the given waveform analysis on the extracted given waveform data corresponding to the inspiratory phase.

7. The waveform analysis apparatus according to claim 6, wherein the apparatus further comprising a comprehensive analyzing section that comprehensively analyzes the vital sign waveform data based on an analysis result obtained by the first waveform analyzing section, and an analysis result obtained by the second waveform analyzing section.

8. The waveform analysis apparatus according to claim 6, wherein the vital sign waveform data are electrocardiogram waveform data.

9. The waveform analysis apparatus according to claim 7, wherein the vital sign waveform data are electrocardiogram waveform data.

10. The waveform analysis apparatus according to claim 8, wherein the extracting section extracts waveform data of a T-wave corresponding to the expiratory phase, and waveform data of a T-wave corresponding to the inspiratory phase from the electrocardiogram waveform data,
the first waveform analyzing section performs a TWA analysis on the extracted waveform data of the T-wave corresponding to the expiratory phase, and
the second waveform analyzing section performs the TWA analysis on the extracted waveform data of the T-wave corresponding to the inspiratory phase.

11. The waveform analysis apparatus according to claim 9, wherein the extracting section extracts waveform data of a T-wave corresponding to the expiratory phase, and waveform data of a T-wave corresponding to the inspiratory phase from the electrocardiogram waveform data,
the first waveform analyzing section performs a TWA analysis on the extracted waveform data of the T-wave corresponding to the expiratory phase, and
the second waveform analyzing section performs the TWA analysis on the extracted waveform data of the T-wave corresponding to the inspiratory phase.

12. The waveform analysis apparatus according to claim 8, wherein the extracting section extracts waveform data of a J-wave corresponding to the expiratory phase, and waveform data of a J-wave corresponding to the inspiratory phase from the electrocardiogram waveform data,
the first waveform analyzing section performs a J-wave analysis on the extracted waveform data of the J-wave corresponding to the expiratory phase, and
the second waveform analyzing section performs the J-wave analysis on the extracted waveform data of the J-wave corresponding to the inspiratory phase.

13. The waveform analysis apparatus according to claim 9, wherein the extracting section extracts waveform data of a J-wave corresponding to the expiratory phase, and waveform data of a J-wave corresponding to the inspiratory phase from the electrocardiogram waveform data,
the first waveform analyzing section performs a J-wave analysis on the extracted waveform data of the J-wave corresponding to the expiratory phase, and
the second waveform analyzing section performs the J-wave analysis on the extracted waveform data of the J-wave corresponding to the inspiratory phase.

14. The waveform analysis apparatus according to claim 6, wherein the vital sign waveform data are pulsewave waveform data,
the extracting section extracts waveform data of a pulse wave corresponding to the expiratory phase, and waveform data of a pulse wave corresponding to the inspiratory phase from the pulse wave waveform data,
the first waveform analyzing section performs an alternans analysis on the extracted waveform data of the pulse wave corresponding to the expiratory phase, and
the second waveform analyzing section performs the alternans analysis on the extracted waveform data of the pulse wave corresponding to the inspiratory phase.

15. The waveform analysis apparatus according to claim 7, wherein the vital sign waveform data are pulse wave waveform data,
the extracting section extracts waveform data of a pulse wave corresponding to the expiratory phase, and waveform data of a pulse wave corresponding to the inspiratory phase from the pulse wave waveform in data,
the first waveform analyzing section performs an alternans analysis on the extracted waveform data of the pulse wave corresponding to the expiratory phase, and
the second waveform analyzing section performs the alternans analysis on the extracted waveform data of the pulse wave corresponding to the inspiratory phase.

16. A computer readable storage medium storing a waveform analysis program causing a computer to perform a process to analyze vital sign waveform data by using respiratory waveform data which are synchronized with the vital sign waveform data, the process comprising:
acquiring the vital sign waveform data and the respiratory waveform data;
determining an expiratory phase and an inspiratory phase from the respiratory waveform data;
extracting given waveform data corresponding to the expiratory phase, and given waveform data corresponding to the inspiratory phase from the vital sign waveform data;
performing a first waveform analysis in which a given waveform analysis is applied to the extracted given waveform data corresponding to the expiratory phase; and
performing a second waveform analyzing in Which the given waveform analysis is applied to the extracted given waveform data corresponding to the inspiratory phase.

17. The computer readable storage medium according to claim 16, wherein the vital sign waveform data are electrocardiogram waveform data.

18. The computer readable storage medium according to claim 17, wherein, in the extraction, waveform data of a T-wave corresponding to the expiratory phase, and waveform data of a T-wave corresponding to the inspiratory phase are extracted from the electrocardiogram waveform data,
in the first waveform analysis, a TWA analysis is performed on the extracted waveform data of the T-wave corresponding to the expiratory phase, and,
in the second waveform analysis, the TWA analysis is performed on the extracted waveform data of the T-wave corresponding to the inspiratory phase.

19. The computer readable storage medium according to claim 17, wherein, in the extraction, waveform data of a J-wave corresponding to the expiratory phase, and waveform data of a J-wave corresponding to the inspiratory phase are extracted from the electrocardiogram waveform data,
in the first waveform analysis, a J-wave analysis is performed on the extracted waveform data of the J-wave corresponding to the expiratory phase, and,
in the second waveform analysis, the J-wave analysis is performed on the extracted waveform data of the J-wave corresponding to the inspiratory phase.

20. The computer readable storage medium according to claim 1, wherein the vital sign waveform data are pulse wave waveform data,
in the extraction, waveform data of a pulse wave corresponding to the expiratory phase, and waveform data of a pulse wave corresponding to the inspiratory phase are extracted from the pulse wave waveform data,
in the first waveform analysis, an alternans analysis is performed on the extracted waveform data of the pulse wave corresponding to the expiratory phase, and,
in the second waveform analysis, the alternans analysis is performed on the extracted waveform data of the pulse wave corresponding to the inspiratory phase.
